(19) United States
(12) Patent Application Publication
(10) Pub. No.: US 2026/0157825 A1
(43) Pub. Date: Jun. 11, 2026

(54) APPARATUSES FOR GENERATING A THREE-DIMENSIONAL BITE RAISING MODEL, METHODS FOR GENERATING A THREE-DIMENSIONAL BITE RAISER, AND NON-TRANSITORY COMPUTER-READABLE STORAGE MEDIA THEREOF

(71) Applicant: CHUN-YEN CHANG, Taipei City (TW)

(72) Inventor: CHUN-YEN CHANG, Taipei City (TW)

(21) Appl. No.: 19/217,270

(22) Filed: May 23, 2025

(30) Foreign Application Priority Data
Dec. 10, 2024 (TW) 113147951

(51) Int. Cl. A61C 9/00; A61B 5/00; A61C 19/05

(52) U.S. Cl. CPC A61C 9/0053; A61B 5/4547; A61C 19/05

(57) ABSTRACT

An apparatus for generating a bite raising model identifies target tooth of the first jaw that first contacts the second jaw based on centric relation, determines a planned overbite position based on an incisal edge of target tooth and planned overbite value, defines a planned occlusal plane based on planned overbite position and functional cusp positions of two first molars, identifies an occlusal contact point on planned occlusal plane based on centric relation, generates a base model having a contact surface adapted to contact consecutive teeth, and generates a platform model extending from the base model, the platform model having an occlusal surface aligned with the planned occlusal plane and covering the occlusal contact point. The bite raising model may be transmitted to a three-dimensional printer to fabricate a bite raiser.

### CROSS REFERENCE TO RELATED APPLICATION AND CLAIM OF PRIORITY

This application claims the benefit under 35 USC § 119 of Taiwan Patent Application No. 113147951 filed on Dec. 10, 2024, in the Taiwan Intellectual Property Office, the entire disclosure of which is incorporated herein by reference for all purposes.

### BACKGROUND

### 1. Field of the Invention

The present invention relates to apparatuses for generating a bite raising model (also referred to as a three-dimensional bite raising model), methods for generating a bite raiser (also referred to as a three-dimensional bite raiser), and non-transitory computer-readable storage media thereof. To be more specific, the present invention relates to apparatuses for generating a bite raising model, methods for generating a bite raiser, and non-transitory computer-readable storage media thereof that involve three-dimensional scanning, three-dimensional modeling, and three-dimensional printing technologies.

### 2. Background Art

Deep overbite, whether arising from congenital anomalies or acquired factors such as tooth wear, trauma, or tooth loss, is often characterized by a deficiency in vertical occlusal space. This vertical insufficiency represents a major challenge not only to successful full-mouth reconstruction but also to functional occlusal rehabilitation. Clinically, bite-raising techniques are commonly employed to manage this condition and can be broadly classified into two categories: fixed bite raisers and removable bite raisers.

Fixed bite raisers involve the direct bonding of a simulated platform onto the palatal or lingual surface of a single tooth. These devices are typically fabricated from materials such as metal alloys, acrylic blocks, or light-cured composite resins. Despite their widespread use, such devices present several limitations, irrespective of the material. Firstly, retention is primarily dependent on chemical bonding between the adhesive and the tooth enamel, which becomes unreliable when anterior teeth are restored with zirconia, metal, porcelain, or temporary polymethyl methacrylate (PMMA) crowns—or in cases of edentulism. Secondly, the occlusal forces are concentrated entirely on the root of a single tooth, thereby increasing the risk of periapical stress and related complications. Thirdly, fixed bite raisers are incompatible with removable orthodontic aligner systems. Fourthly, when bonded to the palatal or lingual surfaces, improper positioning and adhesive overflow into the gingival sulcus may occur, potentially triggering periodontal inflammation.

Additional material-specific drawbacks are also observed. Metallic bite raisers may induce iatrogenic wear on opposing dentition due to pronounced differences in flexural strength and elastic modulus compared to natural tooth structures. Moreover, their lack of customization restricts clinical flexibility. Bite raisers made from light-cured composite resins are susceptible to fracture or debonding under vertical or lateral loads, often necessitating extensive chairside adjustments, and may cause mucosal irritation or ulceration if not properly contoured and polished.

Removable bite raisers—typically realized as extruded features (e.g., bite ramps) on clear aligners—offer an alternative strategy. However, this approach is also associated with multiple limitations. The inherent elasticity of aligner materials renders them prone to deformation under occlusal loading, resulting in compromised structural integrity and positional instability. Moreover, proprietary software used in aligner design often restricts the dimensional capability of extruded features, particularly when overbite or overjet exceeds 3 mm. As a result, these structures may inadequately transmit vertical forces through the tooth's center of resistance. Furthermore, discrepancies between the predicted and actual occlusal parameters during treatment may necessitate the remanufacturing of the entire aligner set. Patient non-compliance with prescribed wear schedules further exacerbates the problem, diminishing the likelihood of achieving the intended occlusal height.

Given these limitations, there is a compelling need for a novel bite-raising solution that allows for individualized customization based on a patient's dental arch morphology and crown morphology. An ideal system would be capable of withstanding functional occlusal forces while mitigating the mechanical and biological drawbacks associated with conventional bonding techniques.

### SUMMARY

An objective of the present invention is to provide an apparatus for generating a bite raising model. The apparatus includes a storage device, an input interface, and a processor electrically connected to the storage device and the input interface. The storage device is configured to store an intraoral three-dimensional scanning graphic file, wherein the intraoral three-dimensional scanning graphic file includes data representing a dentition of a first jaw and a centric relation between the first jaw and a second jaw. The input interface is configured to receive a planned overbite value. The processor is configured to perform the following operations: (a) identify, from the dentition, at least one target tooth of the first jaw that first contacts the second jaw based on the centric relation, (b) determine a planned overbite position based on an incisal edge of the at least one target tooth and the planned overbite value, (c) define a planned occlusal plane based on the planned overbite position and a functional cusp position on a palatal or lingual side of each of the two first molars recorded in the dentition, (d) identify an occlusal contact point on the planned occlusal plane based on the centric relation, (e) generate a base model of the bite raising model according to the dentition, wherein the base model includes a contact surface adapted to contact, on palatal or lingual side, a plurality of consecutive teeth recorded in the dentition, and the consecutive teeth at least include the at least one target tooth, and (f) generate a platform model of the bite raising model based on the planned occlusal plane and the dentition, wherein the platform model protrudes toward the palatal or lingual side from a portion of the base model corresponding to the at least one target tooth, and wherein an occlusal surface of the platform model is aligned with the planned occlusal plane and covers the occlusal contact point.

In some embodiments, the at least one target tooth is at least one of the two central incisors and the two lateral incisors recorded in the dentition.

In some embodiments, the consecutive teeth that the base model can contact include two central incisors, two lateral incisors, and two canines recorded in the dentition.

In some embodiments, the two canines include a first canine and a second canine, and the base model extends from a first palatal or lingual marginal ridge of the first canine to a second palatal or lingual marginal ridge of the second canine transversely.

In some embodiments, each of the two canines has an incisal edge, a region extends from the incisal edge of each canine toward a root, and the base model does not contact the regions.

In some embodiments, the planned overbite position is at a position away from the incisal edge of the at least one target tooth toward a root for the planned overbite value.

In some embodiments, the input interface is further configured to receive an extension length value, and on the occlusal surface, a distance from the occlusal contact point to an edge of the occlusal surface is equivalent to the extension length value.

Another objective of the present invention is to provide a method for generating a bite raiser, and the method is executed by an electronic computing device. The electronic computing device stores an intraoral three-dimensional scanning graphic file and a planned overbite value, wherein the intraoral three-dimensional scanning graphic file includes data representing a dentition of a first jaw and a centric relation between the first jaw and a second jaw. The method includes the following steps: (a) identifying, from the dentition, at least one target tooth of the first jaw that first contact the second jaw based on the centric relation, (b) determining a planned overbite position based on an incisal edge of the at least one target tooth and the planned overbite value, (c) defining a planned occlusal plane based on the planned overbite position and a functional cusp position on a palatal or lingual side of each of two first molars recorded in the dentition, (d) identifying an occlusal contact point on the planned occlusal plane based on the centric relation, (e) generating a base model of a bite raising model according to the dentition, wherein the base model includes a contact surface adapted to contact, on palatal or lingual side, a plurality of consecutive teeth recorded in the dentition, and the consecutive teeth at least include the at least one target tooth, (f) generating a platform model of the bite raising model according to the planned occlusal plane and the dentition, wherein the platform model protrudes toward the palatal or lingual side from a portion of the base model corresponding to the at least one target tooth, and wherein an occlusal surface of the platform model is aligned with the planned occlusal plane and covers the occlusal contact point, and (g) transmitting the bite raising model to a three-dimensional printer so that the three-dimensional printer prints out the bite raiser according to the bite raising model.

In some embodiments, the at least one target tooth is at least one of two central incisors and two lateral incisors recorded in the dentition.

In some embodiments, the consecutive teeth that the base model can contact include two central incisors, two lateral incisors, and two canines recorded in the dentition.

In some embodiments, the two canines include a first canine and a second canine, and the base model extends from a first palatal or lingual marginal ridge of the first canine to a second palatal or lingual marginal ridge of the second canine transversely.

In some embodiments, each of the two canines has an incisal edge, a region extends from the incisal edge of each canine toward a root, and the base model does not contact the regions.

In some embodiments, the planned overbite position is at a position away from the incisal edge of the at least one target tooth toward a root for the planned overbite value.

In some embodiments, the method further includes the step of receiving an extension length value by the electronic computing device, wherein on the occlusal surface, a distance from the occlusal contact point to an edge of the occlusal surface is equivalent to the extension length value.

A further objective of the present invention is to provide a non-transitory computer-readable storage medium that stores a computer program including a plurality of codes. After the codes of the computer program are loaded into an electronic computing device, the electronic computing device executes the codes to realize any of the aforementioned methods for generating a bite raiser.

The present invention (at least including apparatuses for generating a bite raising model, methods for generating a bite raiser, and non-transitory computer-readable storage media thereof) utilizes information recorded in a patient's intraoral three-dimensional scanning graphic file (i.e., the dentition of the first jaw and the centric relation between the first jaw and the second jaw. It should be noted that, clinically, the “centric relation” is the “maximum intercuspal position” for most patients. However, for some patients, the “centric relation” and the “maximum intercuspal position” may differ by 1 mm to 2 mm) and the planned overbite value to generate a bite raising model including a base model and a platform model. Based on the bite raising model, a three-dimensional printer may print out a bite raiser to be used in practice. Since the present invention refers to the patient's intraoral three-dimensional scanning graphic file when generating the bite raising model, it allows customization according to the dentition (e.g., crown morphology) of the patient. In addition, since the contact surface of the base model of the bite raising model is used to contact a plurality of consecutive teeth on palatal or lingual side, the bite raiser printed out based on the bite raising model can sustain a greater occlusal force. Furthermore, as the dimension of the bite raising model is derived based on data collection and computation, various discomforts of wearing a printed bite raiser can be avoided.

### BRIEF DESCRIPTION OF THE DRAWINGS

FIG. 1 illustrates a schematic diagram of the architecture of the model generation apparatus 1 according to some embodiments of the present invention.

FIG. 2 illustrates a portion of the teeth of the first jaw and a portion of the teeth of the second jaw recorded in the intraoral three-dimensional scanning graphic file S1.

FIG. 3 illustrates the teeth of the first jaw J1 recorded in the intraoral three-dimensional scanning graphic file S1.

FIG. 4 illustrates the dentition of the first jaw J1 and the bite raising model.

FIG. 5 illustrates a schematic diagram of a specific example of the bite raising model.

FIG. 6 illustrates the main flowchart of the model generation method of the present invention.

### DETAILED DESCRIPTION

In the following descriptions, apparatuses for generating a bite raising model (also referred to as a three-dimensional bite raising model), methods for generating a bite raiser (also referred to as a three-dimensional bite raiser), and non-transitory computer-readable storage media thereof provided by the present invention will be explained with reference to certain embodiments thereof. However, these embodiments are not intended to limit the present invention to any specific environment, application, or implementations described in these embodiments. Therefore, descriptions of these embodiments are for the purpose of illustration rather than to limit the scope of the present invention. It should be noted that elements unrelated to the present invention are omitted from description in the following embodiments and the attached drawings to avoid obscuring the understanding of the present invention. In addition, the dimensions of elements and dimension scales between individual elements in the attached drawings are provided only for ease of depiction and illustration but not to limit the scope of the present invention. Furthermore, unless otherwise specified, the terms “a,” “the,” and similar terms used in this specification and the claims should be understood to include both singular and plural forms.

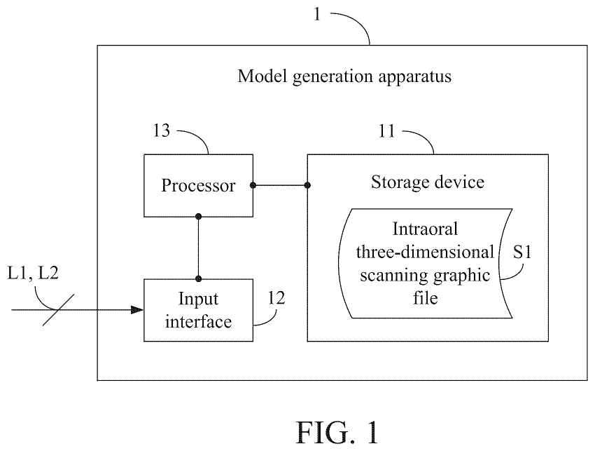

FIG. 1 illustrates a schematic diagram of the architecture of the model generation apparatus 1 (i.e., the apparatus for generating a bite raising model, which is three-dimensional) according to some embodiments of the present invention. The model generation apparatus 1 includes a storage device 11, an input interface 12, and a processor 13, wherein the processor 13 is electrically connected to the storage device 11 and the input interface 12.

The storage device 11 may include one or more of the following elements: memory, hardware disk, and other non-transitory storage media, circuits, or devices capable of storing data known to those of ordinary skill in the art. The input interface 12 may include one or more of the following elements: keyboard, mouse, touch screen, trackball, voice converter and any other devices that allow users to input commands and/or data known to those of ordinary skill in the art. The processor 13 may include one or more of the following elements: processor, central processing unit, microprocessor, digital signal processor, graphic processing unit, and any other computing devices known to those of ordinary skill in the art.

For patients who need to improve malocclusion and/or require full-mouth reconstruction, the model generation apparatus 1 can generate a digital bite raising model for them. The bite raising model includes a base model and a platform model. The bite raising model can be printed out as a bite raiser by a three-dimensional printer. A doctor then adheres the bite raiser to the patients'teeth on the palatal or lingual side to correct the malocclusion. The model generation apparatus 1 is described in detail as follows.

The storage device 11 stores an intraoral three-dimensional scanning graphic file S1. The intraoral three-dimensional scanning graphic file S1 is a file obtained after an operator (e.g., a doctor, a nurse, a dental practitioner) scans a patient's oral cavity using an intraoral scanner. For a patient, the intraoral scanner performs intraoral three-dimensional scanning to establish the patient's dentition and crown morphology and then store these information as an intraoral three-dimensional scanning graphic file S1. To be more specific, the intraoral three-dimensional scanning graphic file S1 includes data representing the dentition of a first jaw, the dentition of a second jaw, and the centric relation between the first jaw and the second jaw. The centric relation is the only stable relationship between the first jaw and the second jaw. It should be noted that, clinically, the “centric relation” is the “maximum intercuspal position” for most patients; however, for some patients, the “centric relation” and the “maximum intercuspal position” may differ by 1 mm to 2 mm. It is also noted that the dentition of the first jaw and the dentition of the second jaw each records information related to a plurality of teeth. A person having ordinary skill in the art shall be familiar with the file and its content obtained after using an intraoral scanner to scan a patient's oral cavity, hence the details are not further described herein.

The present invention does not limit the file format of the intraoral three-dimensional scanning graphic file S1, but for example, the file format of the intraoral three-dimensional scanning graphic file S1 may follow the specification of the Standard Tessellation Language (STL). In addition, the present invention does not limit how the model generation apparatus obtain the intraoral three-dimensional scanning file S1. For example, the model generation apparatus 1 may be electrically connected to the intraoral scanner to receive the intraoral three-dimensional scanning file S1. As another example, the model generation apparatus may include a network interface and/or an input-output interface to receive the intraoral three-dimensional scanning file S1.

The input interface 12 receives a planned overbite value L1, which is the vertical overlap distance or ratio of the central incisal edge of the first jaw and the central incisal edge of the second jaw. The planned overbite value L1 is determined by the doctor according to the patient's occlusal function and/or aesthetic requirements. Generally, the planned overbite value L1 may be set to about 1.0 mm to 2.5 mm in the direction from the central incisal edge to the root of the first jaw, or to a value corresponding to 20% to 40% overlap between the upper and lower crowns.

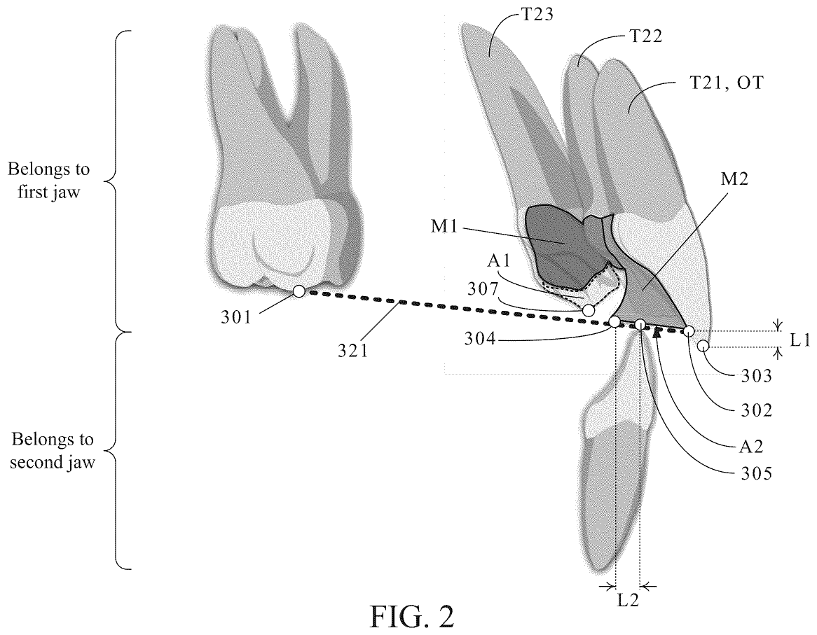

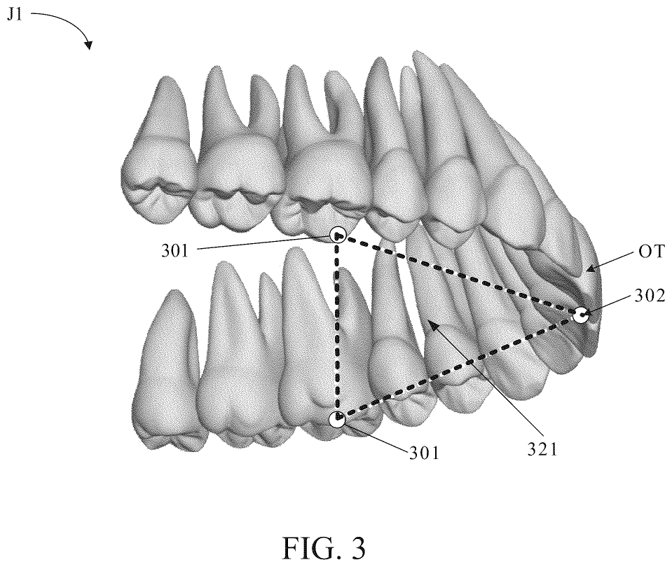

Hereinafter, how the model generation apparatus 1 generates a bite raising model for a patient according to the intraoral three-dimensional scanning file S1 and the planned overbite value L1 is described in details. For ease of understanding and as an example, the first jaw and the second jaw refer to the upper jaw and the lower jaw respectively in the following description; however, it should be understood that the first jaw and the second jaw may refer to the lower jaw and the upper jaw respectively in other examples. In addition, please also refer to the specific examples shown in FIG. 2 and FIG. 3 in the following description, wherein FIG. 2 illustrates a portion of the teeth of the first jaw and a portion of the teeth of the second jaw recorded in the intraoral three-dimensional scanning graphic file S1 and FIG. 3 illustrates the teeth of the first jaw recorded in the intraoral three-dimensional scanning graphic file S1. It should be noted that the specific examples illustrated in FIG. 2 and FIG. 3 are not intended to limit the scope of the present invention.

The processor 13 identifies, from the dentition, the target tooth OT of the first jaw that first contacts the second jaw based on the centric relation (not shown) recorded in the intraoral three-dimensional scanning file S1. It should be noted that in some embodiments, the tooth (or teeth) of the first jaw that first contacts the second jaw may be at least one of the two central incisors and two lateral incisors of the first jaw. In the specific examples illustrated in FIG. 2 and FIG. 3, the target tooth OT is one of the central incisors in the dentition of the first jaw J1.

After the target tooth OT is identified, the processor 13 determines a planned overbite position 302 based on the planned overbite value L1 and the incisal edge 303 of the target tooth OT. To be more specific, the planned overbite position 302 is at a position away from the incisal edge 303 of the target tooth OT toward the root for the planned overbite value L1. After that, the processor 13 defines a planned occlusal plane 321 based on the planned overbite position 302 and the functional cusp position 301 on the palatal or lingual side of each of the two first molars recorded in the dentition of the first jaw. In FIG. 2, the planned occlusal plane 321 is shown only as a dotted line due to the viewing angle. As to FIG. 3, the planned occlusal plane 321, formed by the planned overbite position 302 and the functional cusp positions 301 on the palatal or lingual side of the two first molars, is shown clearly as a dotted triangle. In addition, the processor 13 identifies an occlusal contact point 305 of the first jaw and the second jaw on the planned occlusal plane 321 based on the centric relation recorded in the intraoral three-dimensional scanning file S1.

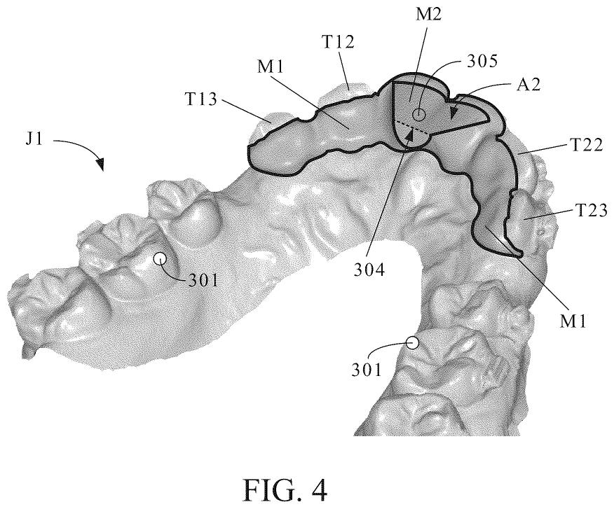

As described above, the bite raising model generated by the model generation apparatus 1 includes a base model and a platform model. Hereinafter, the operation of the processor 13 in generating the base model and the features of the based model will now be described. Please also refer to FIG. 2 and FIG. 4 for the following description, wherein the specific example shown in FIG. 4 illustrates the dentition information of the first jaw J1 and the bite raising model.

The processor 13 generates the base model of the bite raising model according to the dentition of the first jaw. The base model is generated in a way that the base model includes a contact surface adapted to contact, on the palatal or lingual side, a plurality of consecutive teeth recorded in the dentition of the first jaw, and the consecutive teeth include at least the target tooth OT in the dentition. For example, the processor 13 may generate the base model by tracking the anatomical structure of the palatal or lingual side according to the dentition of the first jaw J1.

In the specific examples shown in FIG. 2 and FIG. 4, the consecutive teeth that the base model can contact include two central incisors, two lateral incisors, and two canines. It should be noted that FIG. 2 only shows the central incisor T21, the lateral incisor T22, and the canine T23 on the left side, and FIG. 4 only shows two lateral incisors T21 and T22 and two canines T13 and T23 due to the viewing angle. In the specific example illustrated in FIG. 4, the base model M1 extends from the palatal or lingual marginal ridge of the canine T23 to the palatal or lingual marginal ridge of the canine T13 transversely (i.e., extends in a transverse direction).

Furthermore, in some embodiments, if the consecutive teeth that the base model M1 may contact include two canines, a region extends from the incisal edge of each canine toward the root, and the base model M1 does not contact these regions. It should be noted that in some other embodiments, a region may also extend from the incisal edge of each lateral incisors toward the root, and the base model M1 does not contact these regions. For example, a height of each region may preset to a value from 2.5 mm to 3.0 mm.

In the specific example illustrated in FIG. 2, a region A1 extends from the incisal edge 307 of the canine T13 toward the root. The region A1 is on the palatal or lingual side of the canine T13, and the base model M1 does not contact the region A1 on palatal or lingual side of the canine T13. From FIG. 4, one can also observe that there are regions from the incisal edges of the canines T13 and T23 toward the roots that do not contact the base model M1 and, also, there are regions from the incisal edges of the lateral incisors T12 and T22 toward the roots that do not contact the base model M1.

In some embodiments, the edge of the adhesive surface (i.e., the surface that contacts the teeth) of the base model M1 terminates at the gingival zenith level in the direction toward the root. Therefore, when adhering the bite raiser fabricated (i.e., printed out) based on the bite raising model to the patient's crown surface at a later stage, compression of intraoral soft tissue or gingival sulcus can be avoided.

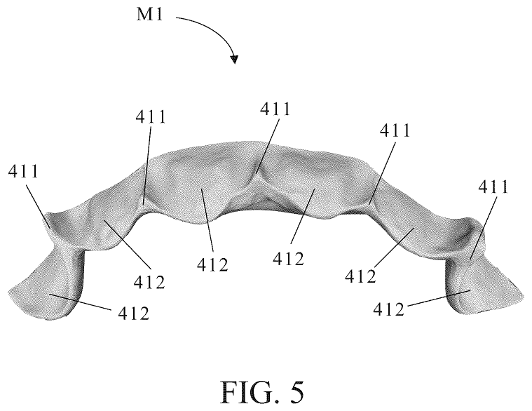

FIG. 5 illustrates another specific example of the base model M1 of the bite raising model. In this specific example, the base model M1 includes six tooth positions 412 (corresponding to two central incisors, two lateral incisors, and two canines) and five protruding undercuts 411 on the contact surface, wherein the protruding undercuts 411 may extend into the inter-proximal surfaces adequately to increase the area of the adhesive surface of the base model M1. It should be understood that the number and the shape of the tooth positions and the protruding undercuts of the base model M1 may differ in different embodiments.

Hereinafter, the operation of the processor 13 in generating the platform model of the bite raising model and the features of the platform model will now be described. Please also refer to both FIG. 2 and FIG. 4 for the following description.

The processor 13 generates the platform model M2 based on the planned occlusal plane 321 and the dentition of the first jaw J1. The platform model M2 protrudes toward the palatal or lingual side from a portion of the base model M1 corresponding to the target tooth OT, the occlusal surface A2 of the platform model M2 is aligned with the planned occlusal plane 321 and covers the occlusal contact point 305. In the specific examples shown in FIG. 2 and FIG. 4, the occlusal surface A2 of the platform model M2 extends from the planned overbite position 302 toward the functional cusp positions 301 on the palatal or lingual side of the two first molars.

In some embodiments, the input interface 12 may receive an extension length value L2. In those embodiments, on occlusal surface A2, the distance from the occlusal contact point 305 to the edge 304 of the occlusal surface A2 is the extension length value L2. That is, the extent that the platform model M2 protrudes toward the palatal or lingual side is limited, the extension of the occlusal surface A2 on the planned occlusal plane 321 toward the palatal or lingual side will exceed the occlusal contact point 305 but the exceeding part is limited within the extension length value L2. By setting this limitation, when adhering the bite raiser fabricated (i.e., printed out) based on the bite raising model to the patient's crown surface at a later stage, the patient will not feel uncomfortable due to the extension length of the platform model M2 will not be excessively long.

In some embodiments, the dentition of the first jaw and/or the detention of the second jaw in the intraoral three-dimensional scanning graphic file may have missing tooth. In those embodiments, to complete the dentition of the first jaw and the dentition of the second jaw, the input interface 12 may receive required parameters and then the processor 13 may supplement the missing tooth temporarily by mirroring according to the parameters or retrieving data from existing database according to the parameters. Afterwards, the processor 13 executes the aforementioned operations.

As described above, the bite raising model generated by the model generation apparatus 1 can be printed out as a bite raiser by the three-dimensional printer. The doctor then adheres the biter raiser to the palatal or lingual side of the patient's teeth to improve malocclusion. The present invention does not limit how the three-dimensional printer obtains the bite raising model. For example, the model generation apparatus 1 may include a network interface and/or an input-output interface to output the biter raising model.

In some embodiments, the three-dimensional printer may use polymethyl methacrylate (PMMA) as three-dimensional printing material and fabricate the bite raiser according to the bite raising model. Using three-dimensional printing material has the advantages of high cost-effectiveness, high accuracy, high safety, and compliance with human biocompatibility standards. In some embodiments, three-dimensional printers with technologies such as stereolithography apparatus (SLA), digital light processing (DLP), and liquid crystal display printing (LCD) may be used to achieve high precision requirement. In some embodiments, any new type of materials and output technologies that meet the human biocompatibility standards and known to those of ordinary skill in the art may be adopted to fabricate the bite raiser according to the bite raising model.

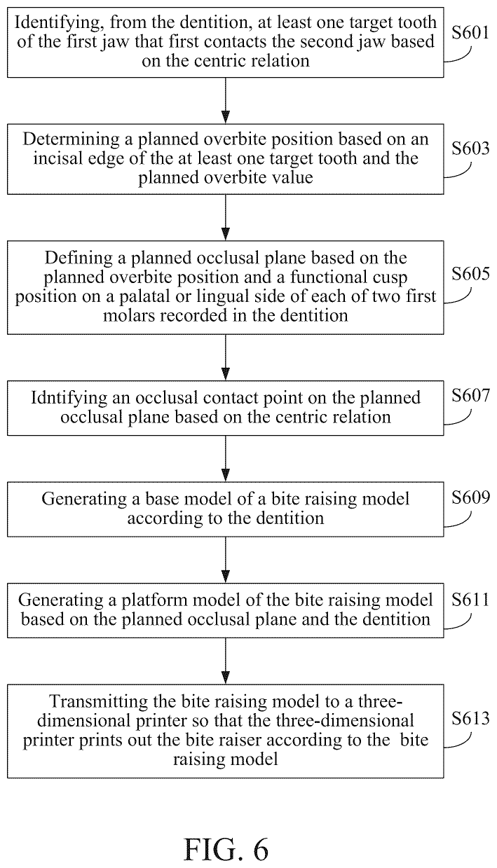

The present invention also provides methods for generating a bite raiser (hereinafter referred as “model generation method”). The bite raiser is also referred to as a three-dimensional bite raiser. A model generation method is executed by an electronic computing device (e.g., model generation apparatus 1). The electronic computing device stores an intraoral three-dimensional scanning graphic file and a planned overbite value, wherein the intraoral three-dimensional scanning graphic file includes data representing a dentition of a first jaw and a centric relation between the first jaw and a second jaw. The main flowchart of a model generation method of the present invention is illustrated in FIG. 6, which includes step S601 to step S613.

The step S601 is executed by the electronic computing device for identifying, from the dentition, at least one target tooth of the first jaw that first contact with the second jaw based on the centric relation. In some embodiments, the at least one target tooth is at least one of two central incisors and two lateral incisors recorded in the dentition.

The step S603 is executed by the electronic computing device for determining a planned overbite position based on an incisal edge of the at least one target tooth and the planned overbite value. In some embodiments, the planned overbite position is at a position away from the incisal edge of the at least one target tooth toward a root for the planned overbite value.

The step S605 is executed by the electronic computing device for defining a planned occlusal plane based on the planned overbite position and a functional cusp position on a palatal or lingual side of each of two first molars recorded in the dentition.

The step S607 is executed by the electronic computing device for identifying an occlusal contact point on the planned occlusal plane based on the centric relation.

The step S609 is executed by the electronic computing device for generating a base model of a bite raising model according to the dentition, wherein a contact surface of the base model is used to contact, on the palatal or lingual side, a plurality of consecutive teeth recorded in the dentition, and the consecutive teeth at least include the at least one target tooth.

In some embodiments, the consecutive teeth that the base model can contact include two central incisors, two lateral incisors, and two canines recorded in the dentition. In some embodiments, the two canines include a first canine and a second canine, and the base model extends from a first palatal or lingual marginal ridge of the first canine to a second palatal or lingual marginal ridge of the second canine transversely. In some embodiments, each of the two canines has an incisal edge, a region extends from the incisal edge of each canine toward a root, and the base model does not contact the regions.

The step S611 is executed by the electronic computing device for generating a platform model of the bite raising model based on the planned occlusal plane and the dentition, wherein the platform model protrudes toward palatal or lingual side from a portion of the base model corresponding to the at least one target tooth, and wherein an occlusal surface of the platform model is aligned with the planned occlusal plane and covers the occlusal contact point.

In some embodiments, the model generation method further includes a step for receiving an extension length value by the electronic computing device. In these embodiments, on the occlusal surface, a distance from the occlusal contact point to an edge of the occlusal surface is equivalent to the extension length value.

The step S613 is executed by the electronic computing device for transmitting the bite raising model to a three-dimensional printer so that the three-dimensional printer prints out the bite raiser according to the bite raising model.

In addition to the aforementioned steps, the model generation method provided by the present invention can also execute other steps so as to have the aforementioned functions of the model generation apparatus 1 and deliver the same technical effects as the model generation apparatus 1. A person having ordinary skill in the art shall readily understand how the model generation method provided by the present invention may execute the steps based on the various embodiments described above, have the same functions, and deliver the same technical effects, hence the details are not further described.

The model generation method described in each of the aforementioned embodiments may be implemented as a computer program having a plurality of codes. The computer program may be stored in a non-transitory computer readable storage medium. After the codes included in the computer program are loaded into an electronic computing device (e.g., the model generation apparatus 1), the computer program executes the model generation method as described in each of the aforementioned embodiments. The non-transitory computer readable medium may be an electronic product, such as a read only memory (ROM), a flash memory, a floppy disk, a hard disk, a compact disk (CD), a digital versatile disc (DVD), a mobile disk, a database accessible to network, or any other storage medium having the same function and well-known to a person having ordinary skill in the art.

It shall be appreciated that, in the specification and the claims of the present invention, some terms (including jaw and canine) are preceded by the terms “first” or “second.” It is noted that the terms “first” and “second” are used only for distinguishing different terms. If the order of these terms is not specified or the order of the terms cannot be derived from the context, the order of these terms is not limited by the preceded “first” or “second.”

According to the above descriptions, the present invention (at least including the apparatuses for generating a bite raising model, the methods for generating a bite raiser, and the non-transitory computer readable storage media thereof) utilizes the information (i.e., the dentition of the first jaw and the centric relation between the first jaw and the second jaw) recorded in a patient's intraoral three-dimensional scanning graphic file and the planned overbite value to generate a bite raising model including a base model and a platform model. Based on the bite raising model, a three-dimensional printer may fabricate (i.e., print out) a bite raiser to be used in practice. Since the present invention refers to the patient's three-dimensional scanning graphic file when generating the bite raising model, it allows customization according to the dentition (e.g., crown morphology) of the patient. In addition, since the contact surface of the base model of the bite raising model is used to contact a plurality of consecutive teeth on palatal or lingual side, the bite raiser printed out based on the bite raising model can sustain a greater occlusal force. Furthermore, the dimension of the bite raising model is arranged ingeniously so that various discomforts of wearing a printed bite raiser can be avoided.

The aforementioned embodiments are used to illustrate some implementation of the present invention and explain the features of the present invention, and not to limit the scope of the present invention. Any changes or equivalent arrangements that can be easily accomplished by persons having ordinary skill in the art are considered within the scope of the present invention, and the protection scope of the present invention is based on the claim of the present invention.

What is claimed is:

1. An apparatus for generating a bite raising model, comprising:
a storage device configured to store an intraoral three-dimensional scanning graphic file, wherein the intraoral three-dimensional scanning graphic file includes data representing a dentition of a first jaw and a centric relation between the first jaw and a second jaw;
an input interface configured to receive a planned overbite value; and
a processor electrically connected to the storage device and the input interface, the processor configured to:
identify, from the dentition, at least one target tooth of the first jaw that first contacts the second jaw based on the centric relation;
determine a planned overbite position based on an incisal edge of the at least one target tooth and the planned overbite value;
define a planned occlusal plane based on the planned overbite position and a functional cusp position on a palatal or lingual side of each of two first molars recorded in the dentition;
identify an occlusal contact point on the planned occlusal plane based on the centric relation;
generate a base model of the bite raising model according to the dentition, wherein the base model includes a contact surface adapted to contact, on the palatal or lingual side, a plurality of consecutive teeth recorded in the dentition, the consecutive teeth at least comprise the at least one target tooth; and
generate a platform model of the bite raising model based on the planned occlusal plane and the dentition, wherein the platform model protrudes toward the palatal or lingual side from a portion of the base model corresponding to the at least one target tooth, and wherein an occlusal surface of the platform model is aligned with the planned occlusal plane and covers the occlusal contact point.

2. The apparatus of claim 1, wherein the at least one target tooth is at least one of two central incisors and two lateral incisors recorded in the dentition.

3. The apparatus of claim 1, wherein the consecutive teeth that the base model can contact comprise two central incisors, two lateral incisors, and two canines recorded in the dentition.

4. The apparatus of claim 3, wherein the two canines comprise a first canine and a second canine, and the base model extends from a first palatal or lingual marginal ridge of the first canine to a second palatal or lingual marginal ridge of the second canine transversely.

5. The apparatus of claim 3, wherein each of the two canines has an incisal edge, a region extends from the incisal edge of each canine toward a root, and the base model does not contact the regions.

6. The apparatus of claim 1, wherein the planned overbite position is at a position away from the incisal edge of the at least one target tooth toward a root for the planned overbite value.

7. The apparatus of claim 1, wherein the input interface is further configured to receive an extension length value, and on the occlusal surface, a distance from the occlusal contact point to an edge of the occlusal surface is equivalent to the extension length value.

8. A method for generating a bite raiser, the method being executed by an electronic computing device, the electronic computing device storing an intraoral three-dimensional scanning graphic file and a planned overbite value, the intraoral three-dimensional scanning graphic file including data representing a dentition of a first jaw and a centric relation between the first jaw and a second jaw, the method comprising:
identifying, from the dentition, at least one target tooth of the first jaw that first contacts the second jaw based on the centric relation;
determining a planned overbite position based on an incisal edge of the at least one target tooth and the planned overbite value;
defining a planned occlusal plane based on the planned overbite position and a functional cusp position on a palatal or lingual side of each of two first molars recorded in the dentition;
identifying an occlusal contact point on the planned occlusal plane based on the centric relation;
generating a base model of a bite raising model according to the dentition, wherein the base model includes a contact surface adapted to contact, on the palatal or lingual side, a plurality of consecutive teeth recorded in the dentition, and the consecutive teeth at least comprise the at least one target tooth;
generating a platform model of the bite raising model based on the planned occlusal plane and the dentition, wherein the platform model protrudes toward the palatal or lingual side from a portion of the base model corresponding to the at least one target tooth, and wherein an occlusal surface of the platform model is aligned with the planned occlusal plane and covers the occlusal contact point; and
transmitting the bite raising model to a three-dimensional printer so that the three-dimensional printer prints out the bite raiser according to the bite raising model.

9. The method of claim 8, wherein the at least one target tooth is at least one of two central incisors and two lateral incisors recorded in the dentition.

10. The method of claim 8, wherein the consecutive teeth that the base model can contact comprise two central incisors, two lateral incisors, and two canines recorded in the dentition.

11. The method of claim 10, wherein the two canines comprise a first canine and a second canine, and the base model extends from a first palatal or lingual marginal ridge of the first canine to a second palatal or lingual marginal ridge of the second canine transversely.

12. The method of claim 10, wherein each of the two canines has an incisal edge, a region extends from the incisal edge of each canine toward a root, and the base model does not contact the regions.

13. The method of claim 8, wherein the planned overbite position is at a position away from the incisal edge of the at least one target tooth toward a root for the planned overbite value.

14. The method of claim 8, further comprising:
receiving an extension length value by the electronic computing device,
wherein on the occlusal surface, a distance from the occlusal contact point to an edge of the occlusal surface is equivalent to the extension length value.

15. A non-transitory computer readable storage medium storing a computer program comprising a plurality of codes, the computer program executing an operation for generating a bite raiser after the codes are loaded into an electronic computing device, the electronic computing device storing an intraoral three-dimensional scanning graphic file and a planned overbite value, the intraoral three-dimensional scanning graphic file including data representing a dentition of a first jaw and a centric relation between the first jaw and a second jaw, the operation comprising:
identifying, from the dentition, at least one target tooth of the first jaw that first contacts the second jaw based on the centric relation;
determining a planned overbite position based on an incisal edge of the at least one target tooth and the planned overbite value;
defining a planned occlusal plane based on the planned overbite position and a functional cusp position on a palatal or lingual side of each of two first molars recorded in the dentition;
identifying an occlusal contact point on the planned occlusal plane based on the centric relation;
generating a base model of a bite raising model according to the dentition, wherein the base model includes a contact surface adapted to contact, on the palatal or lingual side, a plurality of consecutive teeth recorded in the dentition, and the consecutive teeth at least comprise the at least one target tooth;
generating a platform model of the bite raising model based on the planned occlusal plane and the dentition, wherein the platform model protrudes toward the palatal or lingual side from a portion of the base model corresponding to the at least one target tooth, and wherein an occlusal surface of the platform model is aligned with the planned occlusal plane and covers the occlusal contact point; and
transmitting the bite raising model to a three-dimensional printer so that the three-dimensional printer prints out the bite raiser according to the bite raising model.

16. The non-transitory computer readable storage medium of claim 15, wherein the at least one target tooth is at least one of two central incisors and two lateral incisors recorded in the dentition.

17. The non-transitory computer readable storage medium of claim 15, wherein the consecutive teeth that the base model can contact comprise two central incisors, two lateral incisors, and two canines recorded in the dentition.

18. The non-transitory computer readable storage medium of claim 17, wherein the two canines comprise a first canine and a second canine, and the base model extends from a first palatal or lingual marginal ridge of the first canine to a second palatal or lingual marginal ridge of the second canine transversely.

19. The non-transitory computer readable storage medium of claim 17, wherein each of the two canines has an incisal edge, a region extends from the incisal edge of each canine toward a root, and the base model does not contact the regions.

20. The non-transitory computer readable storage medium of claim 15, wherein the planned overbite position is at a position away from the incisal edge of the at least one target tooth toward a root for the planned overbite value.
